(19) United States
(12) Patent Application Publication
(10) Pub. No.: US 2011/0229482 A1
(43) Pub. Date: Sep. 22, 2011

(54) Method of inhibiting infection by HCV, other flaviviridae viruses, and any other virus that complexes to low density lipoprotein or to very low density lipoprotein in blood by preventing viral entry into a cell

(76) Inventor: Vincent Agnello, Weston, MA (US)

(21) Appl. No.: 12/380,346

(22) Filed: Feb. 26, 2009

Related U.S. Application Data
(63) Continuation of application No. 11/820,987, filed on Jun. 20, 2007, now abandoned.
Application No. 11/820,987 is a continuation of application No. 10/398,200, filed on Apr. 3, 2003, now abandoned.
(60) Provisional application No. 60/243,594, filed on Oct. 25, 2000.

(51) Int. Cl. A61K 39/395; C12N 5/071; A61K 38/17; A61P 31/12; A61P 31/14

(52) U.S. Cl. 424/139.1; 435/375; 424/172.1; 514/3.7

(57) ABSTRACT

A method of inhibiting infection by Flaviviridae viruses including HCV, GBC/HGV, and BVD in addition to VSV and any other virus capable of forming a complex with a lipoprotein strategies: preventing formation of a complex should one form, altering the conformation of such a complex to prevent its interaction with the cell receptor, blocking the cell receptor for the complex using an antibody to the receptor, blocking binding of the lipoprotein complex to the cell receptor using soluble lipoprotein receptor or fragments thereof, or downregulating the LDL receptor activity of the cells.

### CROSS-REFERENCE TO RELATED APPLICATION

This application claims priority to U.S. Provisional Application Ser. No. 60/243,594 by Agnello et al., filed Oct. 25, 2000, which is hereby incorporated by reference.

### STATEMENT REGARDING FEDERALLY SPONSORED RESEARCH

The research supporting this patent application was funded in part by National Institutes of Health Grant 1R21AI40672.

### BACKGROUND OF THE INVENTION

1. Field of the Invention

The invention relates to a method of inhibiting cellular endocytosis of a virus capable of forming a complex with a lipoprotein. More specifically, the invention relates to a method of inhibiting infection by hepatitis C virus (HCV), by the other Flaviviridae viruses including GB virus C/hepatitis G virus (GBC/HGV) and bovine viral diarrhea virus (BVDV), and by vesicular stomatitis virus (VSV), and by any other virus that can complex to low density lipoprotein (LDL) or very low density lipoprotein (VLDL) by preventing entry of such viruses into a cell via the low density lipoprotein receptor.

2. Description of the Related Art

Hepatitis C virus (HCV) infection is the most prevalent blood borne infection in the Western world and the major cause of chronic hepatitis and hepatocellular carcinoma. As HCV is not readily replicated in cell culture systems, the mechanisms of HCV infection and proliferation have been difficult to elucidate.

An association of HCV infection with mixed cryoglobulinemia has recently been established. Thus, studies of mixed cryoglobulinemia have provided indirect evidence of the mechanism of HCV endocytosis in vivo. Mixed cryoglobulinemia is a systemic vasculitis associated with cold-precipitable immunoglobulins in the blood. A strong association of HCV infection with mixed cryoglobulins has been established (Monti et al. (1995) Q. J. Med. 88, 115-26) and the specific concentration of HCV in type II mixed cryoglobulins that consists of polyclonal IgG and monoclonal IgM has been demonstrated (Agnello et al. (1992) N. Eng. J. Med. 327, 1490-5). It was also shown that very low density lipoprotein (VLDL) is selectively associated with HCV in type II cryoglobulins (Agnello, V., (1997) Springer Semin. Immunopathol. 19, 111-129). In studies on the cutaneous vasculitic lesions in type II cryoglobulinemia using in situ hybridization (ISH), the HCV RNA virion form (positive strand) but not the putative replicative form (negative strand) of the virus was detected in keratinocytes in the cutaneous vasculitic lesions but not in normal skin of the same patients (Agnello et al. (1997) Arthritis Rheum. 40, 2007-15). Furthermore, it was demonstrated that LDL receptors were upregulated on keratinocytes in cutaneous vasculitis lesions compared with normal skin (Agnello et al. (1997) Arthritis Rheum. 40, 2007-15). It was further demonstrated that anti-β lipoprotein precipitates HCV from infected serum (Thomssen et al., (1992) Med. Microbiol. Immunol. 181, 293-300).

The cell receptor for HCV—the putative entry site for HCV into cells—and the mechanism for initiation of infection, however, remained elusive. The CD81 molecule has been proposed as a candidate for the cell receptor ((1998) Science, 282, 938), but the hypothesis remains unconfirmed.

The inability to ascertain the mechanism of HCV cell entry, or endocytosis, hindered the development of drug therapies aimed at prevention of HCV infection. Heretofore, interferon α (IFN) has been the predominant drug used to treat patients with HCV; however, IFN is only partially effective. Specifically, IFN has sustained a viral remission rate of 5-40% when used alone and up to 60% when used in combination with Ribavirin. While the drugs are believed to inhibit replication of the virus, the mechanism of action of both drugs has yet to be specifically defined.

The object of the invention is to identify the mechanism of HCV entry into cells in an effort to develop a method of inhibiting cellular endocytosis of the virus, thereby preventing infection.

### BRIEF SUMMARY OF THE INVENTION

The invention relates to a method of preventing cellular endocytosis of Flaviviridae viruses including HCV, GBC/HGV, and BVDV in addition to VSV and any other virus capable of forming a complex with a lipoprotein by abrogating endocytosis of those viruses via the LDL receptor. Specifically the invention pertains to a method of inhibiting infection by a virus capable of forming a complex with a lipoprotein by preventing formation of a complex between the lipoprotein and virus, dissociating such a complex should one form, altering the conformation of such a complex to prevent its interaction with the cell receptor, blocking the cell receptor for the complex using an antibody to the receptor, blocking binding of the lipoprotein complex to the cell receptor using soluble lipoprotein receptor or fragments thereof, or downregulating the LDL receptor activity of the cells.

### BRIEF DESCRIPTION OF THE DRAWINGS

FIG. 1 demonstrates the specificity of in situ hybridization method for HCV. (A) HEp2 cells 24 hours after inoculation with HCV. (B) HEp2 cells incubated with respiratory syncytial virus. (C) HEp2 cells incubated with adenovirus. Original magnification is 500×.

FIG. 2 demonstrates upregulation of the LDL receptor in G4 cells. (A) The LDL receptor on up-regulated G4 cells visualized using anti-LDL receptor antibody. (B) Uptake of DiI-LDL by G4 cells with upregulated LDL receptors. (C) Inhibition of endocytosis of DiI-LDL by G4 cells. (D) Phase contrast microscopy showing the inhibition of endocytosis of DiI-LDL by G4 cells. Original magnification is 500×.

FIG. 3 demonstrates that HCV is endocytosed via LDL receptors on lymphocytes and hepatoma cells and that the amount of endocytosis correlates with the concentration of LDL receptor in the cell. (A) HCV ISH of HCV-inoculated G4 cells in which the LDL receptor was not upregulated. (B) HCV ISH in G4 cells in which the LDL receptor was upregulated. (C) HCV-infected G4 cells with up-regulated LDL receptor under higher magnification than shown in (B). (D) LDL receptor-upregulated G4 cells pretreated with anti-LDL receptor antibody prior to HCV inoculation. (E) Uptake of HCV by HepG2 hepatoma cell line as shown by ISH. (F) Blocking of the LDL receptor with LDL receptor antibody to prevent endocytosis of HCV. (G) Incubation of Daudi cells with HCV-positive serum. (H) Pretreatment of Daudi cells with PAO to inhibit endocytosis of HCV. Original magnification for (A), (B), (E) and (F) is 500×; for (C), (D), (G) and (H) is 1250×.

FIG. 4 demonstrates that the LDL receptor but not CD81 mediates endocytosis of HCV. (A) and (B) Demonstration of the presence of LDL receptors and the CD81 antigen on Daudi cells by double immunofluorescence technique. (C)-(F) Demonstration of endocytosis of HCV by Daudi cells and inhibition of endocytosis by anti-LDL receptor antibody. (C) Daudi cells not exposed to HCV. (D) Daudi cells inoculated with HCV. (E) Anti-LDL receptor pretreatment of HCV-inoculated Daudi cells. (F) Anti-CD81 pretreatment of HCV-inoculated Daudi cells. Original magnification is 500×.

FIG. 5 compares the effects of soluble LDL receptor and soluble CD81 on the endocytosis of HCV by Daudi cells. (A) Uninoculated control cells. (B) Cells inoculated with HCV. (C) Cells treated with soluble LDL receptor prior to inoculation with HCV. (D) Cells treated with CD81 prior to inoculation with HCV.

FIG. 6 illustrates the LDL receptor. (A) Schematic drawing of the LDL receptor protein and its organization ((1988) J. Biol. Chem., 263, 13282). (B) Amino acid sequence of the LDL receptor.

FIG. 7 shows endocytosis of Flaviviridae viruses other than HCV and by VSV. (A) Infection of BT cell monolayers by cytopathic BVDV as shown by immunofluorescence using anti-BVDV antibody. (B) Infection of BT cells by cytopathic BVDV as shown by phase contrast microscopy. (C) Preincubation of BT cells with anti-LDL receptor antibody as shown by immunofluorescence. (D) Preincubation of BT cells with anti-LDL receptor antibody as shown by phase contrast microscopy. (E) Inoculation of MRC-5 fibroblasts with HSV. (F) Inoculation of MRC-5 cells with HSV after pretreatment with anti-LDL receptor antibody. (G) Infection of MRC-5 cells with VSV. (H) Inoculation of MRC-5 cells with VSV after pretreatment with anti-LDL receptor. (I) Control MRC-5 cells treated with anti-LDL receptor but no virus. Original magnification for (A)-(D) is 500×; for (E)-(I) is 250×.

FIG. 8 compares DiI-LDL endocytosis of MDBK cells to that of CRIB cells. (A) Intense uptake of DiI-LDL by MDBK cells. (B) Phase contrast microscopy of DiI-LDL uptake by MDBK cells. (C) Lack of endocytosis of DiI-LDL demonstrated in CRIB cell line resistant to BVDV infection. (D) Phase contrast microscopy of lack of DiI-LDL uptake by CRIB cells. (E) Infection of MDBK cells with the NY-1 noncytopathic strain of BVDV. (F) Phase contrast microscopy of BY-1 infection of MDBK cells. (G) Inoculation of CRIB cells with the NY-1 strain of BVDV. (H) Phase contrast microscopy of inoculation of CRIB cells with NY-1 strain of BVDV. Original magnification is 500×.

FIG. 9 demonstrates inhibition of GB virus. C/HGV (GBC/HGV) infection by anti-LDL receptor antibody. (A) Inoculation of Daudi cells with HGV using in situ hybridization. (B) HGV-inoculation of Daudi cells preincubated with anti-LDL receptor antibody. Original magnification is 1250×.

FIG. 10 demonstrates endocytosis of HCV by hepatocytes of a transgenic mouse expressing human LDL receptor and inhibition of endocytosis by anti-LDL receptor antibody. ISH for HCV RNA; (A) Section of liver biopsy of LDL receptor transgenic mouse after inoculation with HCV; (B) Section of liver biopsy of LDL receptor transgenic mouse pretreated with F(ab)′₂ fragment of anti-LDL receptor prior to inoculation with HCV; (C) Section of liver biopsy of LDL receptor transgenic mouse pretreated with F(ab)′₂ fragment of mouse IgG2b.

FIG. 11 demonstrates endocytosis of HCV by hepatocytes of a transgenic mouse expressing human LDL receptor and compares inhibition of endocytosis by anti-LDL receptor antibody and the 32-amino acid 5^{th} repeat of the first domain of the LDL receptor (the binding domain which binds the LDL receptor binding site on VLDL). ISH for HCV RNA: (A) Section of liver biopsy of a LDL receptor transgenic mouse pretreated with F(ab)′₂ fragment of anti-LDL receptor antibody prior to inoculation with HCV; (B) Section of liver biopsy of a LDL receptor transgenic mouse after inoculation with HCV; (C) Section of liver biopsy of a LDL receptor transgenic mouse pretreated with the 5^{th} repeat peptide prior to inoculation with HCV.

FIG. 12 demonstrates upregulation of LDL receptors by pretreatment with atorvastatin and downregulation by pretreatment with IFN and correlation of endocytosis of HCV with modulation of the LDL receptor in LDL receptor transgenic mice inoculated with HCV. (A) Liver section of control mouse pretreated with saline and inoculated with HCV. The red fluorescent staining corresponds to localization of LDL receptor in hepatocytes not treated with any drug. (B) Liver section of mouse pretreated with IFN and inoculated with HCV. (C) Liver section of mouse pretreated with atorvastatin and then inoculated with HCV. (D) Liver section of same mouse as in (A), where the brown intracellular staining corresponds to localization of HCV in hepatocytes. (E) Liver section of same mouse as in (B), where no ISH signal is detected. (F) Liver section of same mouse as in (C) where the more intense brown staining than seen in (A) indicates increased endocytosis of HCV by hepatocytes. Original magnification is 500×.

FIG. 13 shows elimination of the effect of atorvastatin on endocytosis of HCV by IFN in human LDL receptor transgenic mice. (A) Liver section of a control mouse pretreated with saline and inoculated with HCV. The red fluorescent stain indicates the activity of the LDL receptor. (B) Liver section of mouse pretreated with atorvastatin and inoculated with HCV. (C) Liver section of mouse pretreated with atorvastatin and IFN and inoculated with HCV. The absence of fluorescence compared to that seen in (A) and (B) indicates that the upregulation of the LDL receptor manifested in (B) was negated by IFN, confirming that the two drugs have opposite effects on the expression of the LDL receptor. (D) Liver section of same mouse as in (A) where the brown intracellular staining corresponds to localization of HCV in hepatocytes. (E) Liver section of same mouse as in (B) where the more intense brown staining than seen in (A) indicates increased endocytosis of HCV. (F) Liver section of same mouse as in (C) where the lack of an ISH signal indicates lack of endocytosis of HCV, thus confirming the negation of the effect of atorvastatin by IFN.

FIG. 14 compares the effects of IFN (A) and F(ab)′₂ mouse mAb anti-LDL receptor (B) on serum HCV and LDL cholesterol concentration on the same chimpanzee.

### DETAILED DESCRIPTION OF THE INVENTION

An object of the invention is to elucidate the mechanism of endocytosis of HCV in an effort to identify therapeutic strategies to prevent HCV infection.

The inventor conclusively confirmed that HCV and other members of the Flaviviridae virus family are endocytosed by the LDL receptor. Direct evidence supporting this conclusion is provided by LDL-receptor inhibition studies using anti-LDL receptor antibody and known biochemical inhibitors of LDL endocytosis which prevent endocytosis of HCV. It was further determined that CD81 does not mediate entry of HCV into the cell. Furthermore, while the LDL receptor is believed to be the main mechanism for cellular entry of HCV, the detection of small amounts of HCV in LDL-deficient fibroblasts inoculated with HCV suggests the existence of an alternative mechanism of HCV endocytosis.

The inventor made the heretofore unknown discovery that endocytosis of HCV via the LDL receptor requires formation of a complex between the virus and VLDL or LDL but not HDL.

In addition to the in vitro studies, in vivo studies using novel human LDL receptor transgenic mice provide a model for studying the mechanism of endocytosis of HCV in an organism and the physiological effects of potential therapeutic agents for preventing HCV. Specifically endocytosis of HCV via the LDL receptor was demonstrated in vivo and the effects of atorvastatin and interferon α have been examined. Interferon has been shown to down-regulate the LDL receptor and thus decreases the endocytosis of HCV.

To determine directly whether interference with LDL receptor mediated endocytosis of HCV inhibits infection, studies were performed in the chimpanzee, the only species other than humans that can be productively infected with HCV. In an HCV-infected chimpanzee, the effect of administration of antibody to the LDL receptor on infection was compared to treatment with IFN, the current drug used for treatment of HCV infection.

The invention will be described in more detail with reference to the examples below without being limited in scope thereto.

### Materials and Methods

Cyclohexanedione, phenylarsine oxide (PAO), heparin sulfate, and ethylene glycol bis(β-aminoethyl ether)-N,N,N′,N′-tetraacetic acid (EGTA) were purchased from Sigma (St. Louis, Mo.); 1,1′-dioctadecyl-3,3,3′,3′-tetramethylindocarbocyanine iodine (DiI) was purchased from Molecular Probes (Eugene, Oreg.). Purified IgG2a mouse monoclonal anti-LDL receptor antibody (C7 clone) was obtained from Oncogene Scientific Products (Cambridge, Mass.). Anti-bovine viral diarrhea virus (BVDV) envelope antibody bovine serum, α49, was provided by Dr. Marc S. Collett (Viro Pharma, Malvern, Pa.). Mouse monoclonal IgG 2a anti-CD-16, anti-CD-19, and anti-transferrin (CD71) were purchased from ImmunoTech (Hialeah, Fla.). Anti-μ was purchased from Jackson Immunoresearch (West Grove, Pa.). Anti-apolipoprotein (αapo) E and A-I were purchased from Cortex (San Leandro, Calif.); αapoB was purchased from Sigma. Purified mouse monoclonal IgG αapo E (1D7), αapo A-I (3G10), and αapo B (4G3) were purchased from the University of Ottawa Heart Institute (Ottawa, Ontario, Canada). F(ab′)₂ preparations of mouse IgG were prepared by treating the mouse monoclonal antibodies from 30 minutes to 10 hours with 3% pepsin (Sigma), pH 3.5 at 37° C. The F(ab′)₂ fragments were isolated by column chromatography using a HR 10/30 Superose 12 column (Pharmacia, Piscataway, N.J.). BVDV-free donor calf serum was purchased form Boyt Veterinary Laboratory (Neosho, Mo.). Potassium bromide density gradient ultracentrifugation was used for preparation of VLDL, LDL, and high density lipoprotein (HDL) from normal sera, and these lipoproteins complexed to HCV from infected sera. The VLDL band, d=0.95-1.006 g/ml, the LDL band, d=1.019-1.063 g/ml, and the HDL band, d=1.063-1.21 g/ml and HCV free of lipoproteins, d>1.21 g/ml, were isolated by aspiration and then dialyzed against Hanks' balanced salt solution (Sigma) containing 0.01% ethylenediaminetetraacetic acid (EDTA). Isolated HCV-VLDL was dissociated to HCV and VLDL by treatment with deoxycholate and fractionated by sucrose density gradient ultracentrifugation as previously described (Prince et al. (1996) J. Viral Hepat. 3, 11-17). The high density HCV fraction, free of lipoproteins, was further fractionated by column chromatography on a lecithin pretreated Superose 6 column (Pharmacia). The peak of HCV present in the void volume was contaminated with small amounts of immunoglobulins that were removed using immobilized rProtein A (Repligen Corp., Needham, Mass.). Immoblotting (dot blots) to detect small amounts of protein was performed as previously described (Agnello et al. (1986) J. Exp. Med. 164, 1809-14). Sensitivity of the assay was 100 pg for IgG and IgM and 200 pg for apolipoproteins B and E. Lipoproteins were quantitated by Lowry assay using commercial kits (Sigma). Highly purified VLDL, LDL, and HDL were purchased from Cortex. Labeling of LDL with DiI was performed as previously described (Arnold et al. (1992) in Lipoprotein Analysis: A practical Approach, eds. Converse, C. A., Skinner, E. R. (IRL Press at Oxford University Press, Oxford, N.Y.), pp. 145-168).

Infected human sera were used as stocks for HCV (3×10⁸ genomic equivalents per milliliter [gE/ml]), GB virus C/hepatitis G virus (GBC/HCV) (2×10⁹ gE/ml), and herpes simplex virus (HSV). BVDV strains NY-1 and National Animal Disease Laboratory (NADL) and vesicular stomatitis virus (VSV), Indiana strain, and respiratory syncytial virus were obtained from American Type Culture Collection (ATCC, Rockville, Md.). Bovine turbinate (BT) and kidney (MDBK) cell lines, HepG2, a hepatoma cell line that is biochemically similar to hepatocytes (Knowles et al. (1980) Science 209, 497-499), Daudi, a B cell lymphoblastoid cell line, the Molt-4 T cell line HEp2, a squamous carcinoma cell line, and normal fibroblasts (MRC-5) were obtained from ATCC. The B lymphocyte lines G4 and E11 were generated from fusion of F3B6 human-mouse heterohybridoma with peripheral B cells from patients with type II cryoglobulinemia and rheumatoid arthritis, respectively. Development of the 35G6 peripheral B cell line, cloned from normal patient, was previously described (Knight et al. (1993) J. Exp. Med. 178, 1903-1911). Four LDL receptor negative cell lines, GM00488C, GM02000F, GM00701B, and GM3040B, were obtained from the National Institute of General Medical Sciences, Human Genetics Mutant Cell Repository, Coriell Institute for Medical Research (Camden, N.J.). Cells resistant to infection with BVDV (CRIB) were provided by Dr. R. O. Donis (University of Nebraska, Lincoln, Nebr.).

LDL Receptor Assays: Cells were cultured in Roswell Park Memorial Institute (RPMI) medium supplemented either with 10% BVDV-free bovine calf serum or with RPMI medium supplemented with 10% lipoprotein-deficient BVDV-free medium to upregulate expression of the LDL receptor. The cells were then washed twice with phosphate-buffered saline (PBS), pH 7.2. Cytospin preparations were made, fixed with acetone, blocked with 5% normal mouse serum, and the LDL receptor visualized by incubating the slides with 5 μg/ml purified IgG 2a monoclonal anti-LDL receptor antibody followed by a 1:50 dilution of fluorescein (FITC)-labeled goat anti-mouse [F(ab)′₂] second antibody (Jackson Immunoresearch, West Grove, Pa.). The demonstration of LDL receptors on adherent cells, MDBK, CRIB, fibroblasts, HepG2, and HEp2 was performed in the same manner except monolayers of cells were cultured and fixed on slides.

Demonstration of endocytosis of DiI-LDL by cells was performed by incubation of 2×10⁵ cells for 2 hours at 37° C. in 5% CO₂ with 20 μg/ml DiI-LDL as previously described (Yen et al. (1994) J. Immunol. Methods 177, 55-67). The cells were washed twice with cold PBS and fixed with 1% buffered paraformaldehyde, and cytospin preparations were made for fluorescent microscopic studies or cells in suspension were analyzed by flow cytometry. Flow cytometric analysis was performed using the Epic XL-MCL cytometer (Coulter Corp., Miami, Fla.) using a 575 BP filter. Nonspecific binding of DiI-LDL was determined using DiI-LDL treated with cyclohexanedione and was subtracted from the DiI-LDL binding to give specific DiI-LDL binding to cultured cells.

HCV RNA and Endocytosis Assays: HCV RNA was detected by reverse transcriptase-polymerase chain reaction (RT-PCR) and in situ hybridization (ISH) assays as previously described (Agnello et al. (1998) Hepatology 28, 573-84). Specificity of the ISH method for HCV was determined by comparing monolayers of human fibroblasts inoculated with either 3×10⁷ gE/ml HCV or dilutions of adenovirus or Rous sarcoma virus (RSV) that produced pathologic changes in cells at 24 hours. After incubation for 24 hours at 37° C., the cultures were assayed for HCV RNA by ISH. The endocytosis assay for HCV was performed as previously described (Agnello et al. (1998) Hepatology 28, 573-84). Five×10⁵ Daudi cells were inoculated with 3×10⁷ gE HCV or GBC/HGV, incubated for 3 hours at 37° C., washed three times, and assayed for intracytoplasmic HCV RNA or GBC/HGV RNA by ISH. RT-PCR and ISH assays for GBC/HGV RNA were performed as previously described (Liu et al. (1999) J. Virol. Methods 79, 149-159). The same methodology was also used for studies with HCV-lipoprotein recombinants. One hundred micrograms each of normal VLDL, LDL, HDL or cyclohexanedione-treated VLDL or LDL were incubated with 10⁶ gE HCV free of lipoproteins and immunoglobulins for 30 minutes at 37° C. and then added to the Daudi cells.

For cytolytic viruses BVDV, NADL, VSV, and HSV, various dilutions of the respective viruses were incubated with monolayers of cells at 4° C. for 1 hour, washed three times with cold PBS, and incubated with fresh medium. Virus dilutions that produced complete cytolysis at 72 hours for BVDV and VSV and 48 hours for HSV were selected. Immunofluorescent detection of intracytoplasmic BVDV was performed on acetone-fixed slides using 1:50 dilutions of anti-BVDV serum and FITC-labeled anti-bovine second antibody. The presence of BVDV in cells was confirmed by RT-PCR using BVDV specific primers (Pellerin et al. (1994) Virology 203, 260-8).

Inhibition Studies: Blocking of LDL receptor with various dilutions of antibodies (anti-LDL receptor, 5-20 μg/ml; control antisera, 5-200 μg/ml) or inhibitors was performed by pretreatment of cells with various concentrations of antisera or inhibitor for 15 minutes at 37° C. and inoculating with virus without washing the cells. Additions of antisera during incubation period were made at 45-minute intervals. Treatment of LDL and VLDL with cyclohexanedione was performed as previously described (Shepherd et al. (1979) J. Lipid Res. 20, 999-1006). In experiments with cytopathic virus, cells were pretreated with 50 μg/ml of anti-LDL receptor antibody for 30 minutes at 4° C. before inoculation with virus at 4° C.

Inhibition of endocytosis by PAO was assessed by pretreating cells with a range of final PAO concentration of 0.1 to 100 μM as previously described (Kreutz et al. (1996) Virus Res. 42, 137-147), and then endocytosis of LDL or HCV was evaluated by the DiI-LDL assay or by HCV-ISH, respectively, as described earlier.

### Example 1

### Endocytosis of HCV Via the LDL Receptor

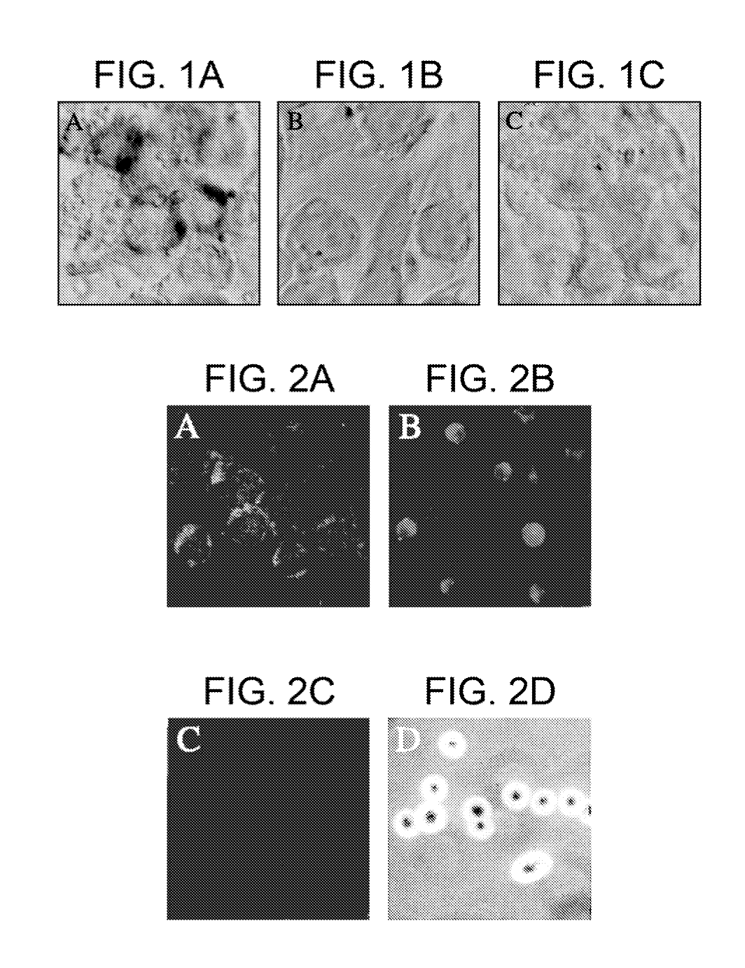

It was previously demonstrated that endocytosis of HCV in vitro correlates with the titer of HCV in the inoculum. The percentage of cells positive for HCV RNA as determined by ISH correlated directly with the number of gE of HCV per cell as determined by RT-PCR (Agnello et al. (1998) Hepatology 28, 573-84). There also was a crude correlation between intensity of ISH staining for HCV RNA and gE HCV per cell by RT-PCR. The specificity of this ISH assay for HCV is shown in FIG. 1. The brown staining of HEp2 cells 24 hours after inoculation with HCV indicates the presence of positive strand HCV (FIG. 1A). In contrast, HEp2 cells incubated with respiratory syncytial virus or adenovirus (FIGS. 1B and 1C, respectively) show no staining for HCV using the same ISH.

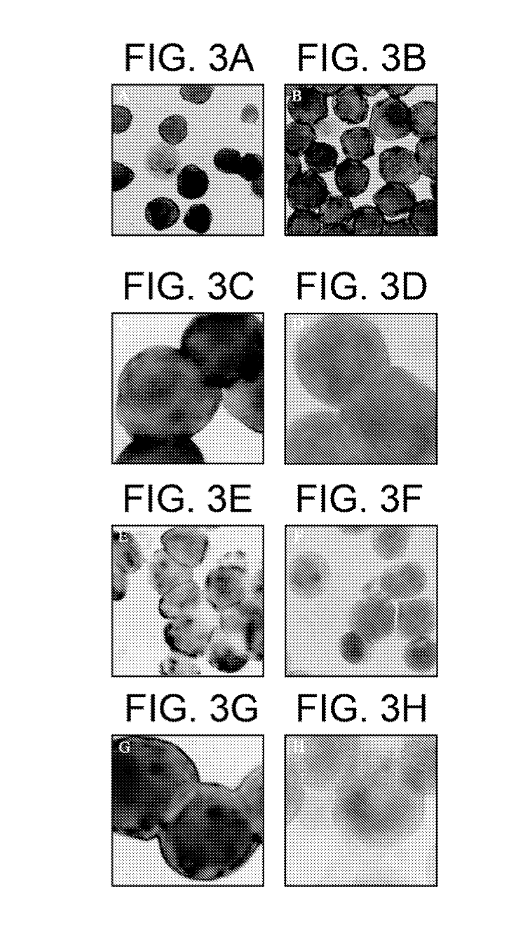

For a further investigation of endocytosis of HCV by cells in vitro, a variety of human cell Cultures were demonstrated to have LDL receptors with the use of anti-LDL receptor antibody or DiI-LDL uptake. These cell lines were then inoculated with a high titer HCV-positive human serum. Intracellular HCV RNA was then detected using ISH. To determine whether endocytosis of HCV correlated with the level of LDL receptor expression on cells, the well-known modulatory effect of lipoproteins on the LDL receptor was used to increase the number of LDL receptors (upregulate) on cells by culturing in lipoprotein deficient media. Relative differences in endocytosis of LDL by various cultured cell lines could be demonstrated by the specific uptake of DiI-LDL. The specific DiI-LDL uptake of HepG2 cells as shown in Table 1 was four times greater than that of the peripheral B cell line, G4, without upregulation. Upregulating these B cells produced a LDL uptake equivalent to that of the HepG2 cells without upregulation. These results were confirmed by immunofluorescent studies using anti-LDL receptor antibody staining and DiI-LDL uptake. Specifically, as shown in FIG. 2A, upregulation of the LDL receptor on G4 cells was visualized with the anti-LDL receptor antibody. FIG. 2B demonstrates the uptake of 5 μg DiI-LDL by the upregulated G4 cells. The uptake of DiI-LDL can be completely inhibited by excess unlabeled LDL as shown in FIG. 2C. As demonstrated using ISH, upregulation of the LDL receptors of G4 cells resulted in 70-80% of cells staining positive for the LDL receptor on HepG2 cells (FIG. 3E) and Daudi cells (FIG. 3G), also known to have higher densities of LDL receptor (Yen et al. (1994) J. Immunol. Methods 177, 55-67).

TABLE 1
Uptake of LDL by Cultured Cells

| Cell line | Culture medium | Treatment | Mean fluorescence |
|---|---|---|---|
| G4 | Routine * | None | 0.8 ± 0.4 |
| G4 | Routine | DiI-LDL | 4 ± 2.0 |
| G4 | Lipoprotein deficient † | DiI-LDL | 16 ± 7 |
| HepG2 | Routine | None | 3 ± 1 |
| HepG2 | Routine | DiI-LDL | 16 ± 5 |

RPMI with 10% fetal bovine serum
† RPMI with lipoprotein deficient serum

The percentage of cells positive for HCV by ISH was shown to correlate with the percentage of cells positive for LDL receptor by immunofluorescence using anti-LDL receptor antibody or DiI-labeled LDL. Endocytosis of HCV by peripheral B cells that showed 5-30% weakly positive cells in routine culture (FIG. 3A) when upregulated showed a percentage of positive cells and an intensity of staining (FIG. 3B) comparable to HepG2 cells (FIG. 3E) and Daudi cells (FIG. 3G) that were each 70-80% positive.

Direct evidence that the LDL-receptor mediated endocytosis of HCV was obtained by inhibiting endocytosis with anti-LDL receptor antibody. The endocytosis of HCV could be inhibited in a dose-dependent manner by preincubating the cells with anti-LDL receptor antibody. At sufficient concentrations of anti-LDL receptor antibody, complete inhibition of endocytosis of the virus could be demonstrated for both G4 and HepG2 cells. As shown in FIG. 3D, HCV endocytosis by upregulated G4 cells is inhibited by pretreatment of the cells with anti-LDL receptor antibody. Likewise, HCV endocytosis by HepG2 cells is blocked by the anti-LDL receptor antibody (FIG. 3F). Similar results were obtained using infected serum or VLDL-HCV complexes isolated from type II cryoglobulins as the inoculum in these experiments. No inhibition was observed with control mouse IgG 2a or antisera to specific cell surface antigens at a concentration up to 20 times the lowest inhibiting concentration of anti-LDL receptor: antisera to μ heavy chain, CD-19, and CD-16 surface antigens on peripheral B cells and Daudi cells and antiserum to transferrin receptor on HepG2 cells did not inhibit the endocytosis of HCV by these cells. Moreover, treatment of Daudi cells with the endocytosis inhibitor PAO at 2 μM concentration completely inhibited the endocytosis of HCV (FIGS. 3G, 3H).

The role of the LDL receptor in the endocytosis of HCV was confirmed by demonstrating competitive inhibition with LDL and VLDL but not HDL, which is known not to bind to the LDL receptor. With use of hepatoma cells (HepG2) or B cells (G4 and E11), 25-100 μg/ml of LDL or VLDL completely inhibited endocytosis of HCV, whereas concentrations of HDL up to 200 μg/ml (a 5-20 and 10-40 fold molar excess over LDL and VLDL, respectively) did not inhibit. Treatment of LDL or VLDL with cyclohexanedione which is known to alter a critical arginine residue in the LDL receptor binding site of apolipoproteins E and B (Shepherd et al. (1979) J. Lipid Res. 20, 999-1006), the main apolipoproteins found in VLDL and LDL, respectively, eliminated the inhibition by LDL or VLDL. Moreover, 25 units/ml of heparin sulfate or 2 μM EGTA inhibited endocytosis of HCV. Both are known inhibitors of LDL receptor endocytosis of lipoprotein (Subramanian et al. (1995) J. Lab. Clin. Med. 125, 479-485).

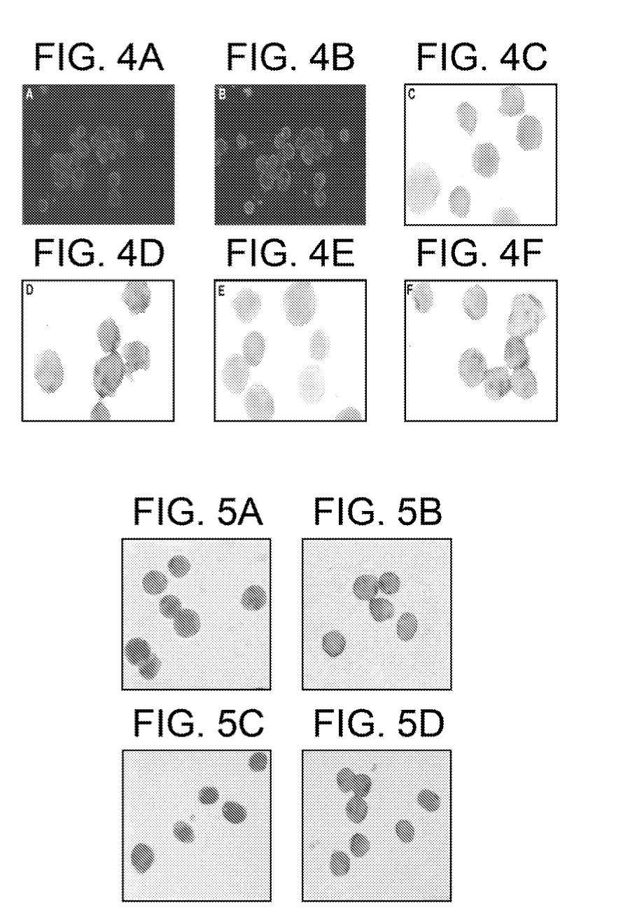

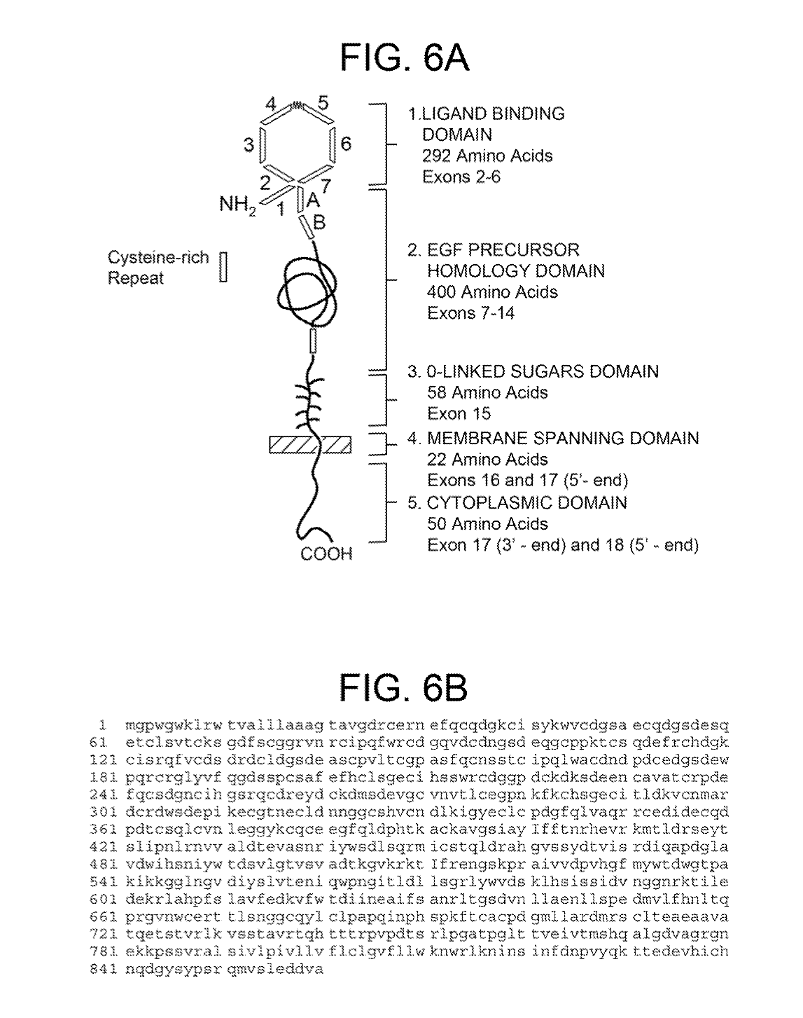

In addition, it was demonstrated that CD81 does not mediate endocytosis of HCV. As shown in FIGS. 4A and 4B, double immunofluorescent microscopy was used to demonstrate the presence of LDL receptors and the CD81 antigen on Daudi cells. Daudi cells were prepared on positively charged slides by cytocentrifugation, fixed in cold acetone-methanol 1:1 for 5 minutes at room temperature (RT), air dried, and blocked at RT for 15 minutes with phosphate buffered saline (PBS), pH 7.4, containing normal mouse serum and 5% normal rabbit serum (blocking buffer). The slides were then incubated with 1:50 dilution JS-64 anti-CD81 mouse monoclonal antibody (ImmunoTech, Hialeah, Fla.) and 1:100 dilution biotinylated rabbit anti-anti-LDL receptor antibody in blocking buffer (RT) for 30 minutes, washed four times with PBS, then incubated with 1:50 dilution of fluorescein isothiocyanine (FITC)-labeled anti-mouse IgG and 1:200 dilution of streptavidin-phycoerythrein in blocking buffer (RT) for 30 minutes in the dark. Negative controls, prepared with the omission of first antibodies, did not show fluorescence, indicating the presence of both LDL receptor and CD81 on this cell line. 10⁵ Daudi cells then were preincubated for 30 minutes at RT with medium alone (FIGS. 4C and 4D), with 5 μg/ml anti-LDL receptor antibody (FIG. 4E), or with 6 μg/ml JS-64 monoclonal anti-CD81 antibody (FIG. 4F) in 12×75 culture tubes in a final volume of 900 μl/tube of RPMI 1640, 10% lipoprotein-deficient donor calf serum containing 2 mM L-glutamine and 25 mM HEPES. 100 μl of an HCV positive serum (3×10⁷ gE/ml) were added to each of three tubes (FIG. 4D-F). The negative control received 100 μl PBS (FIG. 4D). Tubes were further incubated at 37° C. for 3 hours in a culture incubator, washed three times with PBS, and fixed by adding 0.3 ml of 1% buffered formalin (Polysciences Inc., Washington, Pa.). Cells were processed, and ISH was performed as previously described. Cells not exposed to HCV were negative (FIG. 4C). HCV endocytosis was demonstrated in the cells treated only with HCV positive serum (FIG. 4D). Pretreatment of Daudi cells with anti-LDL receptor antibody prior to HCV inoculation inhibited endocytosis of the virus (FIG. 4E), while pretreatment with anti-CD81 antibody did not (FIG. 4F). Inhibition can be achieved using only appropriate soluble fragments of the soluble LDL receptor. For instance, the following fragments of the 841 amino acid LDL receptor (FIG. 6B) should be effective to inhibit endocytosis: amino acids 66-354 of SEQ ID NO:1; amino acids 66-375 of SEQ ID NO:1; amino acids 25-354 of SEQ ID NO:1; amino acids 25-375 of SEQ ID NO:1; amino acids 1-354 of SEQ ID NO: 1; amino acids 1-375 of SEQ ID NO:1; and especially amino acids 193-231 of SEQ ID NO:1, corresponding to the soluble 5^{th} repeat of the LDL receptor.

Moreover, inhibition of endocytosis of HCV by Daudi cells by soluble LDL receptor (SEQ ID NO:1) but not soluble CD81 was demonstrated (FIG. 5). Daudi cells inoculated with HCV and incubated at 37° C. for 2 hours show brown cytoplasmic staining, indicating the presence of the positive strand of HCV (FIG. 5B). Pretreatment with soluble LDL receptor for 30 minutes at 37° C. completely inhibits endocytosis of HCV (FIG. 5C), as evidenced by a lack of staining comparable to that of uninoculated control cells (FIG. 5A). Pretreatment with soluble CD81, however, did not inhibit endocytosis (FIG. 5D).

To determine whether lipoproteins were involved in the endocytosis of HCV, inhibition studies were performed using various previously characterized antisera to apolipoproteins (αapo E ID7 (Weisgraber et al. (1983) J. Biol. Chem. 258, 12348-12354), αapo B 4G3 (Pease et al. (1990) J. Biol. Chem. 265, 553-568), and αapo A-I 3G10 (Marcel et al. (1991) J. Biol. Chem. 266, 3644-3653). F(ab′)₂ fragments were prepared and were used for all of the studies; inhibitory activities of the preparations were tested against DiI-labeled VLDL, LDL, and HDL isolated from a normal serum. Optimum F(ab′)₂ antibody concentrations and conditions for inhibition of endocytosis were determined. Optimum conditions required addition of F(ab′)₂'s after pretreatment during the incubation period, and both αapo E and αapo B were required for maximal inhibition of VLDL endocytosis, whereas αapo B was sufficient for maximal inhibition of LDL endocytosis. Under these conditions, the maximum inhibition of HCV endocytosis achieved was 65%, with the remaining positive cells showing only trace staining. Pretreatment with αapo A-I gave 10% inhibition, with the remaining positive cells showing no decrease of staining compared to the control. The addition of αapo A-I during incubation did not increase inhibition. The finding that both αapo E and αapo B were required and that additional F(ab′)₂'s during the incubation increased inhibition was most likely due to the complexity of VLDL metabolism and dissociation of F(ab′)₂'s binding at 37° C. Hence, it could not be determined whether VLDL alone or both VLDL and LDL mediated endocytosis of HCV. Moreover, because complete inhibition could not be achieved, direct endocytosis of HCV by the LDL receptor could not be excluded.

Endocytosis experiments of isolated HCV-lipoprotein complexes and recombination experiments with HCV and lipoproteins provided more definitive data on the role of lipoproteins in endocytosis of HCV via the LDL receptor. Isolation of HCV by dissociation of HCV-VLDL complexes was unsuccessful; however, density gradient fractionation of a serum containing a high concentration of HCV produced not only HCV lipoproteins fractions but also a high density HCV fraction free of lipoprotein. Immunoglobulins contaminating the latter fraction were removed, providing a “free” HCV fraction for recombinant studies. Comparison of endocytosis of the various fractions is shown in Table 2. The HCV-VLDL and HCV-LDL, but not the HCV-HDL or high density HCV, fractions were endocytosed. Addition of VLDL or LDL but not HDL, isolated from normal serum, to the “free” HCV resulted in restoration of endocytosis. Cyclohexanedione treatment of the VLDL or LDL abrogated the rescue.

TABLE 2
Comparison of Endocytosis of HCV in Lipoprotein
Fractions and the High Density HCV Fraction

| | | | | Endocytosis of |
|---|---|---|---|---|
| | | | Endocytosis of 5 μg | 1 × 10⁶ gE HCV |
| | | Lipoprotein | DiI-labeled fraction | from each fraction |
| | HCV | concentration | mean fluorescence | % cell positive/ |
| Fraction | (gE/ml) | (mg/ml) | (log scale) | intensity of staining |
| VLDL | 2.5 × 10⁶ | 0.48 | 8.49 | 90%, ++ |
| LDL | 2.9 × 10⁶ | 1.47 | 9.98 | 75%, + |
| HDL | 8.3 × 10⁶ | 2.56 | 1.88 | 0 |
| d > 1.21 | 3.9 × 10⁶ | — | — | 0 |

++ Moderately positive
+ Weakly positive

It was further shown that the ligand binding domain of the LDL receptor (FIG. 6A) binds LDL by a specific binding site on apo B100 that includes at least one epitope between residues 2980-3084 or residues 2835-4189 on apo B100 (SEQ. ID. NO: 2). Binding is mediated by at least one epitope between residues 193-232 or 66-375 of the LDL receptor molecule (FIG. 6B) (SEQ ID NO:1). Binding of VLDL to LDL receptor is mediated by a specific binding site on apoE that includes at least one epitope between residues 1-191 and 216-299 on apoE (SEQ ID NO:3). Binding to apoE is mediated by the 5^{th} repeat sequence of the LDL receptor molecule (amino acids 193-231 of SEQ ID NO:1) (FIG. 6A,B). The 1^{st} repeat of the ligand binding domain of the LDL receptor is not involved in binding either LDL or VLDL; however, antibody directed against at least one epitope in the 1^{st} repeat inhibits endocytosis of HCV complexed to LDL or VLDL.

Further studies were performed using the LDL receptor deficient fibroblast cells (Mahley et al. (1977) J. Biol. Chem. 252, 7279-7287). Inoculation of these cells with HCV showed only weak endocytosis that could not be increased with preincubation of cells in lipoprotein deficient medium nor inhibited by anti-LDL receptor antibody. Furthermore, this low level endocytosis could not be competitively inhibited with excess VLDL.

### Example 2

### Replication of Endocytosed HCV

Replication of HCV has been reported in HepG2 (Subramanian et al. (1995) J. Lab. Clin. Med. 125, 479-485) and Daudi (Weisgraber et al. (1983) J. Biol. Chem. 258, 12348-12354) cell cultures. Extended cultures of HepG2, Daudi, and G4 cells were tested serially by ISH for evidence of replication. In the HepG2 cells, only positive-strand HCV was detected in the cells up to 1 week, but at 3 weeks, 85% of the cells contained positive-strand HCV and 65% contained negative strand HCV. At 4 weeks, the cells were negative for HCV. In Daudi cells, only positive strand was detected through day 10, but on days 15 and 20, both positive- and negative-strand genome sequences were present in 80% cells. The cells died in the 4^{th} week of culture. Only the positive strand of HCV was detected in G4 cells up to 1 week; the cells died after 1 week.

### Example 3

### Endocytosis of Other Flaviviridae Viruses

Commercial bovine sera known to be contaminated with the pestivirus, BVDV (Nuttall et al. (1977) Nature, 266, 835-837 and Yanagi et al. (1996) J. Infect. Dis. 174, 1324-1327), were investigated. Human cell lines routinely cultured in media containing bovine serum were found to be positive for intracytoplasmic BVDV by immunofluorescence using anti-BVDV-antibody. The presence of BVDV was confirmed by RT-PCR using BVDV-specific primers. Negative strand BVDV was not detected in cells nonpermissive to infection. BVDV-positive human nonpermissive cells became negative over a 4-week culture period in noncontaminated media. Endocytosis of BVDV by nonpermissive cells could be inhibited completely with anti-LDL receptor antibody but not with the control anti-transferrin receptor antibody.

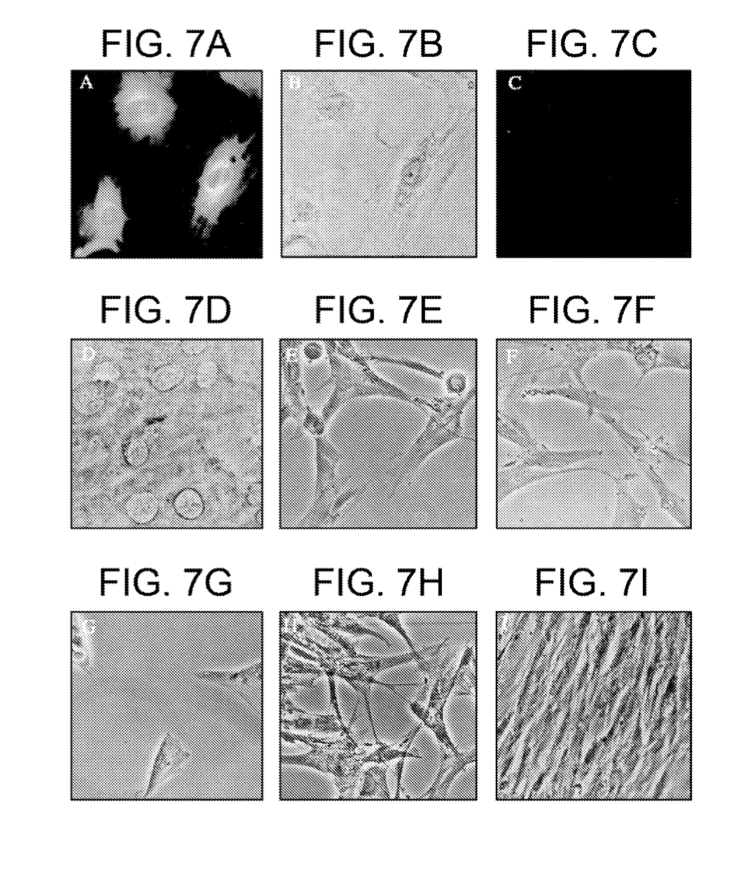

With the use of cytopathic NADL strain of BVDV and permissive cells, BT or bovine kidney (MDBK) cells, anti-LDL receptor antibody but not control antiserum inhibited the cytopathic effect and positive fluorescence at 3 days (FIGS. 7A-D). Immunofluorescence using anti-BVDV antibody demonstrated infection of BT cell monolayers by cytopathic BVDV (NADL strain) after 72 hours of incubation (FIG. 7A; same field is shown in FIG. 7B using phase contrast microscopy). Preincubation of the BT cell monolayers with anti-LDL receptor antibody completely inhibits infection (FIG. 7C; same field shown by phase contrast microscopy in FIG. 7D). Five days after infection, there was complete cytolysis of both the inhibited and control cells. Similar studies using VSV and HSV were performed. No inhibition of infection by anti-LDL receptor was demonstrated for HSV. FIG. 7E shows MRC-5 fibroblasts inoculated with HSV. Widespread cytolysis and destruction of the monolayer were evident after 48 hours in comparison to the control MRC-5 cells treated with the anti-LDL receptor antibody but not with virus (FIG. 7I). Pretreatment of the monolayers with anti-LDL receptor antibody did not prevent cytolysis and death (FIG. 7F). FIG. 7G shows MRC-5 cells inoculated with VSV, resulting in cytopathy and destruction of the monolayer. Pretreatment of the monolayers with anti-LDL receptor antibody showed some inhibition of the destruction of the cells (FIG. 711).

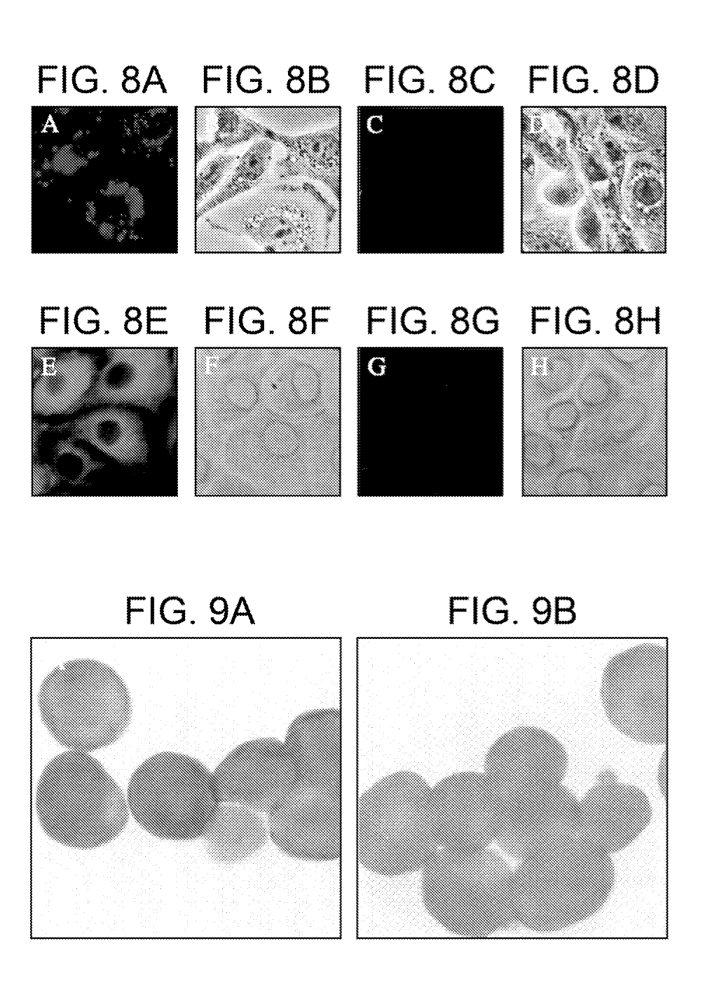

Additional evidence for endocytosis of BVDV by LDL receptor was obtained using a cell line resistant to BVDV, CRIB, that was derived from a permissive bovine kidney cell line MDBK. As illustrated in FIG. 8, the CRIB cells that do not permit entry of BVDV (Flores et al. (1995) Virology, 208, 565-575) also do not endocytose LDL. Specifically, FIG. 8E demonstrates the infection of MDBK cells with the NY-1 noncytopathic strain of BVDV after 72 hours of incubation by immunofluorescence using anti-BVDV antibody (same field shown by phase contrast microscopy in FIG. 8F). In contrast, no BVDV was demonstrated by immunofluorescence in the CRIB cell line inoculated with the virus (FIG. 8G; same field shown by phase contrast microscopy in FIG. 8H). However, the absence of DiI-LDL staining is a more sensitive indication of the absence of LDL receptor and LDL endocytosis because accumulation of DiI-LDL occurs from the rapid turnover of LDL by LDL receptor in the course of cholesterol metabolism. MDBK cells demonstrate an intense uptake of DiI-LDL (FIG. 8A; same field shown using phase contrast microscopy in FIG. 8B). FIG. 8C shows the lack of endocytosis of DiI-LDL by CRIB cells (same field shown using phase contrast microscopy in FIG. 8D).

A third member of the Flaviviridae family, GB virus C/HGV (GBC/HGV) was reported to associate with lipoproteins in the blood (Sato et al. (1996) Biochem. Biophys. Res. Commun., 229, 719-725). Evidence was also obtained for LDL receptor mediated endocytosis of this virus, as illustrated in FIG. 9. Specifically, Daudi cells inoculated with GBC/HGV show the presence of HGV virion in the cytoplasm using ISH specific for this virus (FIG. 9A). Preincubation of the cells with anti-LDL receptor antibody decreased uptake of the virus below the detection limit of ISH (FIG. 9B).

### Example 4

### In Vivo HCV Endocytosis

The LDL receptor controls cholesterol metabolism. Thus, deficiency of the receptor caused by genetic abnormalities cause fatal disease as a result of hypercholestemia. As demonstrated by Examples 1-3, the binding of anti-LDL receptor antibody to the LDL receptor inhibits the endocytosis of HCV in cell culture, but it cannot be determined from these in vitro studies whether the binding of the antibody to the LDL receptor would cause dire physiological consequences in vivo due to hypercholestemia. Also, it cannot be determined if the anti-LDL antibody would be effective in blocking endocytosis of HCV in vivo due to large amounts of to lipoproteins in the circulation that would compete with the antibody for binding sites on the receptor. The anti-LDL receptor antibodies could not be used as a therapeutic agent for the treatment of HCV for the treatment of HCV infection if the antibody itself causes disease.

A human LDL receptor transgenic (hLDLR Tg) mouse was developed to delineate the mechanism of LDL receptor-mediated endocytosis of HCV in vivo and to provide a model for feasibility and toxicity studies on anti-LDL antibody administration in vivo. These mice overexpress the human LDL receptor on hepatocytes. The complete coding region of the ligand binding domain of the human LDL receptor (FIG. 6A,B) under the control of mouse metallothionein-I promoter is present in the transgenic mouse. The version of the human LDL receptor gene inserted in these mice lacks the sequence from intron 5-7 of the complete gene so that it can be distinguished from the mouse LDL receptor gene. The hLDLR gene is expressed in the presence of cadmium (Cd) or zinc (Zn). Thus, overexpression of the gene results in transgenic mice given ZnSO₄ in their drinking water for 7 days, as evidenced by low levels of cholesterol.

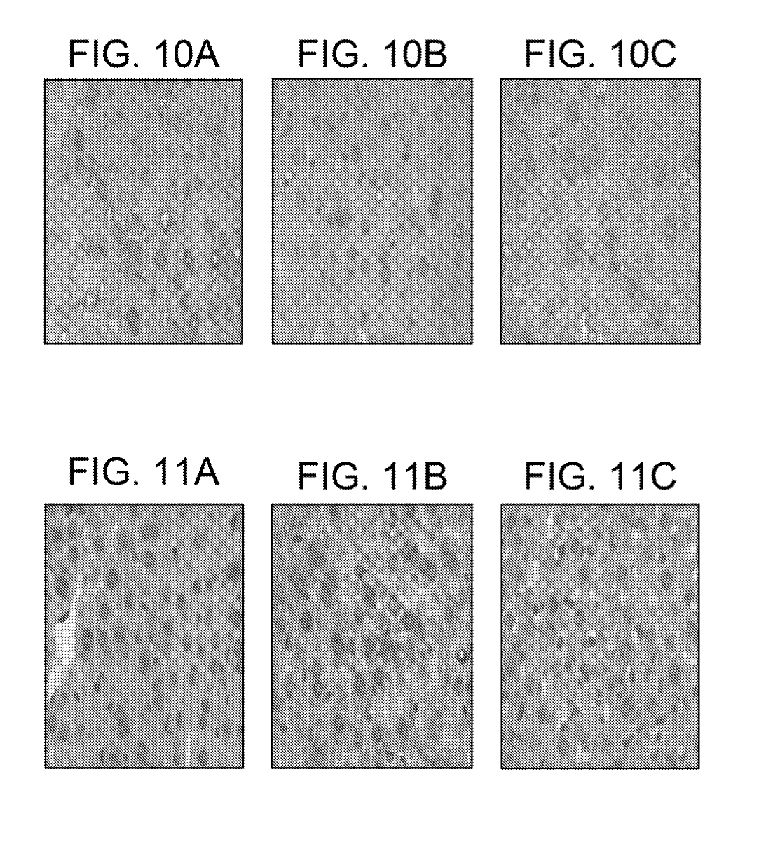

Endocytosis of HCV via the LDL receptors in the hepatocytes in the liver could be demonstrated using the transgenic mice (FIG. 10). ISH for HCV RNA performed on a section of liver biopsy of a hLDLR Tg mouse taken one hour after inoculation of HCV (3.2×10⁶ gE) intraperitoneally showed brown cytoplasmic staining, thus indicating the presence of the virion form (positive strand) of HCV (FIG. 10A). Pretreatment with 1.9 mg F(ab′)₂ fragment of anti-LDL receptor antibody one hour prior to inoculation with HCV completely inhibits endocytosis of HCV (FIG. 10B). Pretreatment with 1.9 mg F(ab′)₂ fragment of mouse IgG2b (Sigma, St. Louis, Mo.) (isotype matched to the mouse anti-LDL receptor antibody) did not inhibit endocytosis of the HCV (FIG. 10C). A F(ab′)₂ fragment of antibody to LDL receptor was used in these mice to eliminate toxicity that may be caused by the Fc portion of the molecule activating complement with binding to antigen. There were no untoward effects on the mice from the F(ab′)₂ antibody when injected at a dose of 1 mg per gram of liver, and there was a minimal elevation of blood cholesterol levels that was transient (Table 3).

TABLE 3
Cholesterol Levels in hLDLR Tg Mice Inoculated with
2 mg F(ab)′₂ anti-LDL receptor monoclonal antibody
Cholesterol Level:

| Mouse | Pretreatment | 1 Hour | 24 Hours | 3 Days | 4 Days |
|---|---|---|---|---|---|
| 1 | 28 | 26 | 39 | 34 | 27 |
| 2 | 33 | — | 31 | 33 | 31 |

Similar inhibition of endocytosis of HCV in hLDLR Tg mice could be obtained using the soluble 5^{th} repeat peptide (FIG. 11). ISH for HCV RNA was used to demonstrate inhibition of endocytosis of HCV by hepatocytes of a hLDLR Tg mouse with the 39 amino acid 5^{th} repeat of the first domain of the LDL receptor, the ligand binding domain. ISH for HCV RNA performed on a section of liver biopsy of a hLDLR Tg mouse taken one hour after inoculation of HCV (5.0×10⁶ gE) intraperitoneally showed brown cytoplasmic staining, thus indicating the presence of the virion form (positive strand) of HCV (FIG. 11B). As demonstrated above, pretreatment with 1.9 mg F(ab)′₂ fragment of anti-LDL receptor antibody one hour prior to inoculation with HCV completely inhibits endocytosis of HCV (FIG. 11A). Likewise, Pretreatment with 1 mg of the 5^{th} repeat peptide one hour prior to inoculation with HCV completely inhibits endocytosis of HCV (FIG. 11C).

### Example 5

### Effects of IFN and Atorvastatin

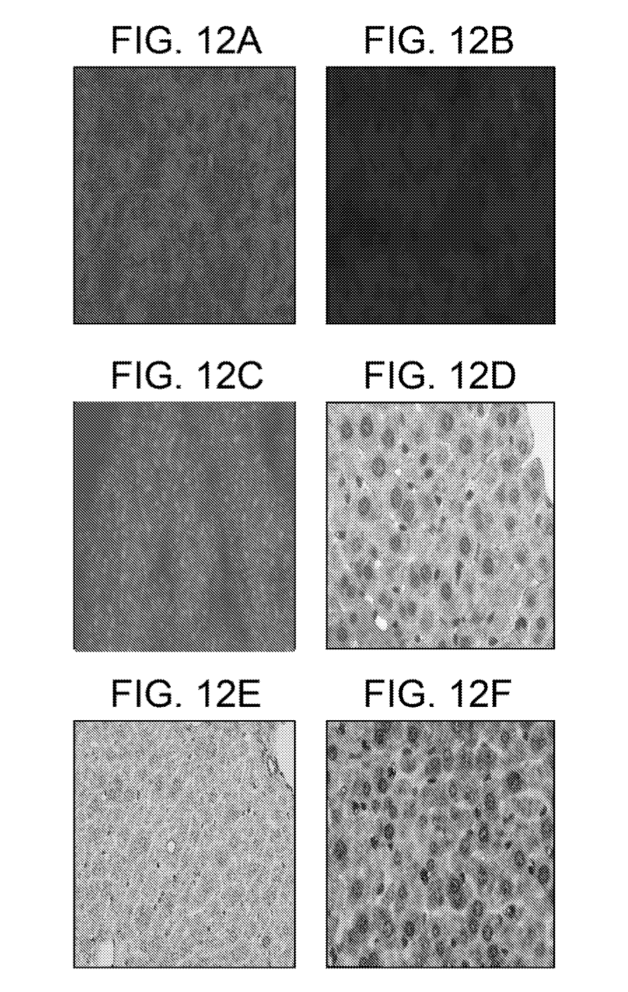

The statin drugs lower blood cholesterol by upregulating the LDL receptor. Administration of atorvastatin to a hLDLR Tg mouse prior to inoculation with HCV increases LDL receptor activity and endocytosis of the virus (FIG. 12). Specifically, the liver section of a Tg mouse pretreated with 0.5 mg atorvastatin and then inoculated with HCV shows upregulation of the LDL receptor by fluorescence staining (FIG. 12C; FIG. 12F shows the increased endocytosis of HCV by hepatocytes of the liver section of the same mouse using ISH for HCV RNA). In contrast, absence of fluorescence by a liver section of a Tg mouse pretreated with 0.1 Mu IFN and then inoculated with HCV indicates downregulation of the LDL receptors (FIG. 12B; FIG. 12E shows no ISH signal indicating a lack of HCV endocytosis by hepatocytes) compared to the control liver section that was pretreated with saline, inoculated with HCV, and sacrificed one hour post-inoculation (FIG. 12A; the brown intracellular staining demonstrated by ISH for HCV RNA in FIG. 12D corresponds to the localization of HCV in hepatocytes). Quantitative HCV studies of the liver of the three mice corresponded with the ISH studies in (D), (E) and (F). The control mouse had 43 HCV gE per mg liver, the IFN pretreated mouse had 9.3 HCV gE per mg liver, and the atorvastatin pretreated mouse had 163 HCV gE per mg liver. Thus, the HCV therapeutic drug IFN downregulates the LDL receptor and decreases endocytosis of HCV.

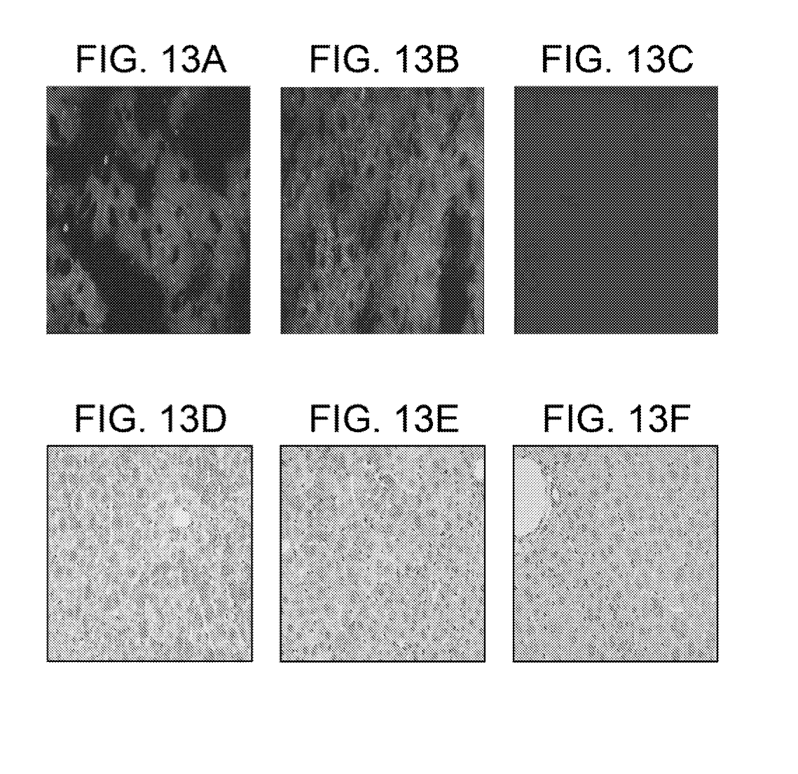

Administration of IFN with atorvastatin negates the upregulation of the LDL receptor and increased endocytosis by atorvastatin (FIG. 13). As demonstrated above, the liver section of a Tg mouse pretreated with 0.5 mg atorvastatin and then inoculated with HCV shows upregulation of the LDL receptor by fluorescence staining (FIG. 13B; FIG. 13E shows the increased endocytosis of HCV by hepatocytes of the liver section of the same mouse using ISH for HCV RNA) when compared to the control mouse pretreated with saline prior to HCV inoculation (FIG. 13A; FIG. 13D shows the same mouse section using ISH for HCV RNA). In contrast, the liver section of a mouse pretreated with both 0.5 mg atorvastatin and 0.5 Mu IFN followed by inoculation with HCV demonstrates that the upregulation of the LDL receptors manifested in FIG. 13B was negated by IFN (FIG. 13C; FIG. 13F shows that no signal for HCV RNA is detected from the same mouse liver section using ISH, indicating lack of endocytosis of HCV). These results confirm that IFN and atorvastatin have opposite effects on the modulation of the LDL receptor and that downregulation of the receptor by a drug can inhibit infection.

### Example 6

### Demonstration of Inhibition of Infection in the Chimpanzee

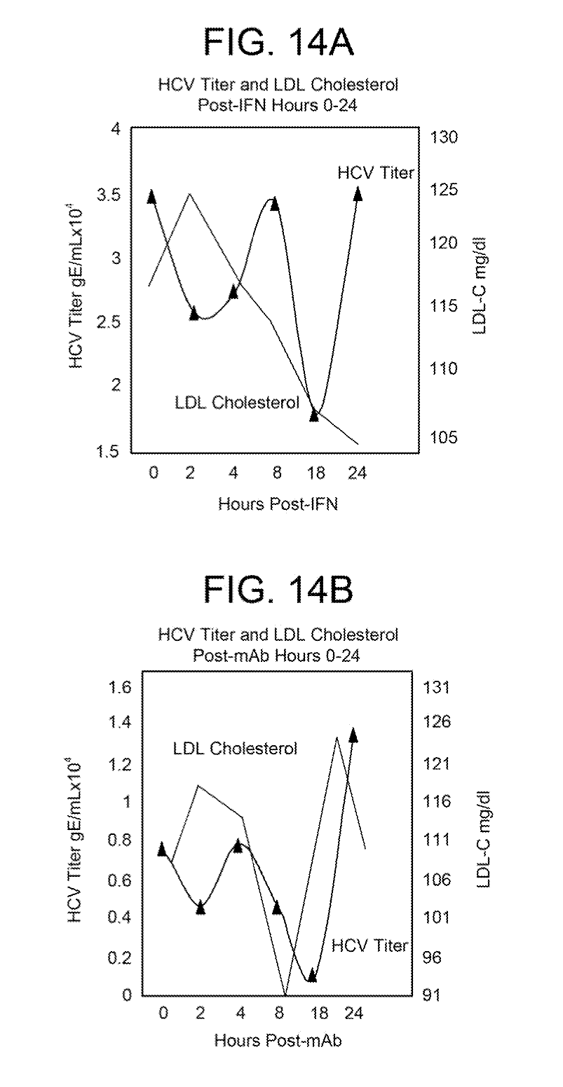

The only species other than humans that can be productively infected with HCV is the chimpanzee. From studies of HCV infected humans, it has been demonstrated that administration of IFN results in a rapid drop of blood HCV concentration within 24 hours following injection of 10 Mu IFN. Comparison of treatment with 10 Mu IFN or F(ab′)₂ antibody to LDL receptor at 25 mg/kg in the same HCV chimpanzee (studies performed one week apart) showed a 50% decline in viremia at 18 hours with IFN (FIG. 14A) compared to an 86% decline at the same point with antibody to the LDL receptor (FIG. 14B). In both studies there was a slight increase in cholesterol that peaked 2 hours post-treatment. Hence, the effect of antibody to LDL receptor on infection appears to be greater than the IFN effect.

The effect of interferon alpha (IFNα), the current therapy for HCV infection, may be mediated in part by the downregulation of LDL receptors. IFNα is known to induce interleukin 1 (IL-1) receptor antagonist (IL-1RA) (Tilg et al. (1993) J. Immunol. 150, 4687-4692), which blocks the IL-1 receptor-mediated stimulation by IL-1. Because IL1 is known to increase LDL receptor activity (Dinarello (1996) Blood 87, 2095-2147), IFNα would indirectly cause a downregulation of LDL receptor activity by stimulating IL-1RA production, thereby decreasing IL-1 receptor-mediated stimulation by IL-1. Other, more direct effects of IFN on the expression of the LDL receptor may also be present.

1.-44. (canceled)

45. A method of inhibiting infection of a cell by a virus having a lipoprotein-binding site and capable of forming a virus-lipoprotein complex by complexing with a lipoprotein having a virus-binding site and a lipoprotein receptor-binding site, said complexing occurring at the respective virus-binding site of the lipoprotein and the lipoprotein-binding site of the virus, said method comprising at least a step selected from the group consisting of:
(a) preventing formation of said lipoprotein complex;
(b) dissociating said virus and said lipoprotein if complexing should occur;
(c) inhibiting the binding of the lipoprotein complex to the cell;
(d) introducing lipase to the cell, wherein said lipase is capable of inducing a conformational change of a virus-lipoprotein complex;
(e) introducing an effective amount of an anti-low density lipoprotein (LDL) receptor antibody (anti-LDLR), wherein said anti-LDLR binds to at least one epitope included in the ligand binding domain of the LDL receptor (amino acids 1-375 of SEQ ID NO:1);
(f) introducing an effective amount of an anti-apolipoprotein(apo)B100 antibody, wherein said anti-apo B100 antibody binds to at least one epitope included in the LDL-receptor binding domain of apo B100 between amino acids 2835 and 4189 of SEQ ID NO:2;
(g) introducing an effective amount of an anti-apoE antibody, wherein said anti-apoE antibody binds at least one epitope included in the LDL receptor binding domain of apo E between amino acids 1-191 or 216-299 of SEQ ID NO:3; and,
(h) introducing an effective amount of a peptide comprising the soluble 5^{th} repeat of the ligand binding domain of the LDL receptor (amino acids 193-231 of SEQ ID NO:1), wherein said peptide fragment binds to the receptor binding domain of at least one of apo B and apo E.

46. The method of claim 45 wherein the virus is a Flaviviridae virus or vesicular stomatitis virus, or other viruses that complex with LDL or VLDL.

47. The method of claim 46 wherein the infection of the cell is inhibited by preventing formation of said lipoprotein complex.

48. The method of claim 47 wherein the formation of said lipoprotein complex is prevented by a ligand or an antibody to a virus-binding site of said lipoprotein.

49. The method of claim 47 wherein the formation of said lipoprotein complex is prevented by a ligand or an antibody to a lipoprotein-binding site of said virus.

50. The method of claim 46 wherein the infection of the cell is inhibited by dissociating said virus and lipoprotein.

51. The method of claim 45(e) wherein said at least one epitope is between amino acids 25-65, or 65-374 of SEQ ID NO:1.

52. The method of claim 51 wherein said at least one epitope is in the first repeat of the ligand binding domain of the LDL receptor included between amino acids 25-65 of SEQ ID NO:1.

53. The method of claim 45(f) wherein said at least one epitope is included in the LDL receptor binding domain of apo B100 between amino acids 2980-3084 of SEQ ID NO:2.

54. The method of claim 45(g) wherein said at least one epitope is included in the LDL receptor binding domain of apo E between amino acids 139-169 of SEQ ID NO:3.

55. The method according to claim 45(h) wherein said peptide comprises amino acids 66-354 of SEQ ID NO:1.

56. The method according to claim 45(h) wherein said peptide comprises amino acids 66-375 of SEQ ID NO:1.

57. The method according to claim 45(h) wherein said peptide comprises amino acids 25-354 of SEQ ID NO:1.

58. The method according to claim 45(h) wherein said peptide comprises amino acids 25-375 of SEQ ID NO:1.

59. The method according to claim 45(h) wherein said peptide comprises amino acids 1-354 of SEQ ID NO:1.

60. The method according to claim 45(h) wherein said peptide comprises amino acids 1-375 of SEQ ID NO:1.

61. The method according to claim 45(h) wherein said peptide comprises soluble LDL receptor (SEQ ID NO:1).

62. A method of treating infection of an organism comprising administering a therapeutically effective amount of at least an agent selected from the group consisting of: (a) anti-apo E antibody; (b) anti-apo B antibody; and (c) a peptide comprising the soluble 5^{th} repeat of the LDL receptor (amino acids 193-231 of SEQ ID NO:1).

63. The method of treating infection of an organism according to claim 62(c), wherein said peptide comprises amino acids 66-354 of SEQ ID NO:1.

64. The method of treating infection of an organism according to claim 62(c), wherein said peptide comprises amino acids 66-375 of SEQ ID NO:1.

65. The method of treating infection of an organism according to claim 62(c), wherein said peptide comprises amino acids 25-354 of SEQ ID NO:1.

66. The method of treating infection of an organism according to claim 62(c), wherein said peptide comprises amino acids 25-375 of SEQ ID NO:1.

67. The method of treating infection of an organism according to claim 62(c), wherein said peptide comprises amino acids 1-354 of SEQ ID NO:1.

68. The method of treating infection of an organism according to claim 62(c), wherein said peptide comprises amino acids 1-375 of SEQ ID NO:1.

69. The method of treating infection of an organism according to claim 62(c), wherein said peptide comprises soluble LDL receptor (SEQ ID NO:1).

70. A method of preventing infection of an organism including mammals by a Flaviviridae virus, vesicular stomatitis virus, or other viruses that complex with LDL or VLDL, comprising at least a step selected from the group consisting of: (a) blocking a lipoprotein receptor on cells of said organism; (b) introducing an effective amount of anti-LDLR antibody that binds to at least one epitope in the ligand binding domain of the LDL receptor (SEQ ID NO:1); and (c) downregulating lipoprotein receptor activity of said cell.

71. The method according to claim 70(a) wherein an antibody to said lipoprotein receptor is used as a blocking agent.

72. A method according to claim 70(b), wherein said anti-LDLR antibody binds to at least one epitope in the first repeat of the ligand binding domain included between amino acids 25-65 of SEQ ID NO:1, and wherein said inhibition of infection occurs without harmful effects on cholesterol metabolism.

73. A pharmaceutical composition for treating infection of an organism comprising a therapeutically effective amount of a peptide comprising the soluble 5^{th} repeat of the ligand binding domain of the LDL receptor (amino acids 193-231 of SEQ ID NO:1) together with a pharmaceutically acceptable carrier or diluent.

74. The pharmaceutical composition according to claim 73, wherein said peptide comprises amino acids 66-354 of SEQ ID NO:1.

75. The pharmaceutical composition according to claim 73, wherein said peptide comprises amino acids 66-375 of SEQ ID NO:1.

76. The pharmaceutical composition according to claim 73, wherein said peptide comprises amino acids 25-354 of SEQ ID NO:1.

77. The pharmaceutical composition according to claim 73, wherein said peptide comprises amino acids 25-375 of SEQ ID NO:1.

78. The pharmaceutical composition according to claim 73, wherein said peptide comprises amino acids 1-354 of SEQ ID NO:1.

79. The pharmaceutical composition according to claim 73, wherein said peptide comprises amino acids 1-375 of SEQ ID NO:1.

80. The pharmaceutical composition according to claim 73, wherein said peptide comprises the soluble LDL receptor (SEQ ID NO:1).


SEQUENCE LISTING (3 sequences)

SEQ ID NO: 1 - protein, 860 aa, Homo sapiens
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYIFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTIFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 2 - protein, 4560 aa, Homo sapiens [sequence omitted]

SEQ ID NO: 3 - protein, 317 aa, Homo sapiens [sequence omitted]